(19) United States
(12) Patent Application Publication
(10) Pub. No.: US 2014/0106336 A1
(43) Pub. Date: Apr. 17, 2014

(54) Detection of Magnetic-Field-Concentrated Analytes in a Lateral Flow Capillary

(71) Applicants: Kent J. Voorhees, Golden, CO (US); Christopher R. Cox, Golden, CO (US); Roy R. Mondesire, Boulder, CO (US)

(72) Inventors: Kent J. Voorhees, Golden, CO (US); Christopher R. Cox, Golden, CO (US); Roy R. Mondesire, Boulder, CO (US)

(73) Assignee: Colorado School of Mines, Golden, CO (US)

(21) Appl. No.: 13/992,547

(22) PCT Filed: Dec. 7, 2011
(86) PCT No.: PCT/US11/63810

§ 371 Date: Dec. 19, 2013

Related U.S. Application Data
(60) Provisional application No. 61/420,411, filed on Dec. 7, 2010.

(51) Int. Cl. G01N 33/569

(52) U.S. Cl. CPC G01N 33/56916; G01N 33/56911
USPC 435/5; 435/287.2

(57) ABSTRACT

The present disclosure generally relates to systems, devices and methods for detecting magnetic-field-concentrated target analytes within a lateral flow capillary.

### CROSS REFERENCE TO RELATED APPLICATION

This application claims the benefit of priority under 35 U.S.C. §119(e) to U.S. Application No. 61/420,411, filed Dec. 7, 2010, titled “MAGNETIC NANOPARTICLE CAPILLARY FLOW,” and which is hereby incorporated by reference in its entirety.

### TECHNICAL FIELD

The present disclosure generally relates to systems, devices and methods for detecting magnetic-field-concentrated target analytes within a lateral flow capillary.

### BACKGROUND

Current methods of species-specific detection and identification of bacteria and other microorganisms are complex, time-consuming, and/or often require expensive specialized equipment and highly trained personnel. Numerous biochemical and genotypic identification methods have been applied to microorganism detection with varied levels of success, but all rely on tedious microbiological culturing practices and/or costly and time-consuming DNA extraction, amplification, and sequencing protocols utilizing highly specialized equipment which render them impractical for deployment as rapid, cost-effective point of care detection and identification methods.

The information included in this Background section of the specification is included for technical reference purposes only and is not to be regarded as subject matter by which the scope of the description is to be bound or as an admission of prior art.

### SUMMARY

The present disclosure is directed to detecting target analytes. A system for detecting a target analyte includes a sample loading section configured to receive a sample, and a capillary, the proximal end of which is fluidly associated with the sample loading section. The system also includes a magnet configured to apply a magnetic field to at least a portion of the capillary, and a detector configured to detect an analyte-magnetic component-reporter molecule complex in the capillary.

A method for detecting target analytes includes mixing a target analyte, a magnetic component configured to bind the target analyte, and a reporter molecule configured to bind the target analyte to form an analyte-magnetic component-reporter molecule complex in a sample. The sample is introduced to a capillary. Applying a magnetic field to at least a portion of the capillary concentrates the complex. The complex can then be detected to determine the presence of the target analyte.

### BRIEF DESCRIPTION OF THE DRAWINGS

The present disclosure, both as to its organization and manner of operation, may be understood by reference to the following description, taken in connection with the accompanying drawings, in which:

FIG. 1 depicts one embodiment of a detection unit;

FIG. 2 depicts one embodiment of analyte preparation and detection;

FIG. 3 depicts an exemplary infectious cycle of a bacterium by a bacteriophage;

FIG. 4 depicts a classic phage growth plot following infection of a single bacterium with a single phage;

FIG. 5A depicts one embodiment of a SERS reporter molecule; FIG. 5B depicts a corresponding Raman spectrum;

FIG. 6 depicts an analyte-magnetic component-reporter molecule complex and its substituents.

FIG. 7 depicts a multiplexed system or device;

FIG. 8A depicts the Raman spectra of various concentrations of E. coli; FIG. 8B depicts a titration curve;

FIG. 9A depicts a detection unit; and FIG. 9B depicts a Raman spectrum.

### DETAILED DESCRIPTION

The present disclosure provides systems, devices, and methods for detecting target analytes. More specifically, the system and device are capable of lateral flow capillary-based analyte transport in combination with the use of anti-analyte antibody-coated magnetic components and anti-analyte antibody-coated reporter molecules. The system and device allow for focused magnetic-based analyte concentration and highly sensitive detection within the capillary. The system and device can be used to detect microorganisms, including bacteria.

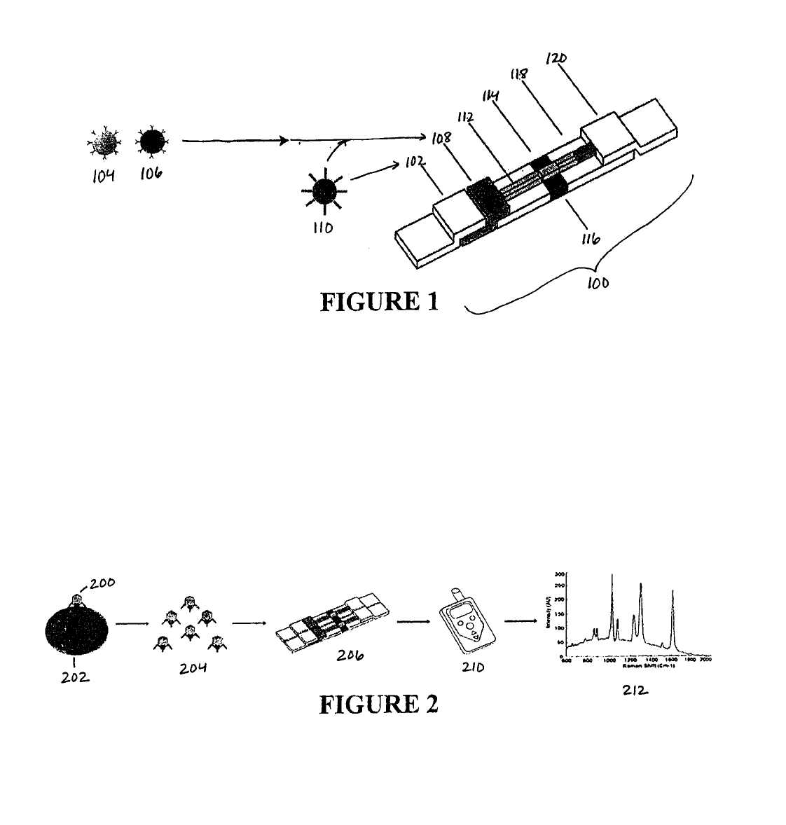

In certain variations, as can be understood from FIG. 1 and described in more detail below, the system or device includes a detection unit 100. A target analyte is prepared and loaded onto a sample loading section 102. A magnetic component 104 and a reporter molecule 106 are loaded onto a magnetic component-reporter molecule loading section 108. Optionally, the magnetic component 104 is loaded onto a magnetic component loading section, which is separate from the reporter molecule loading section onto which a reporter molecule 106 is loaded. Optionally, a control particle 110 can be added to the sample loading section 102 or to the magnetic component-reporter molecule loading section 108.

Continuing with FIG. 1, the sample loading section 102 is fluidly associated with the capillary 112. The target analyte, magnetic component 104, reporter molecule 106, and control particle 110 enter the capillary 112. The sample flows laterally through the capillary 112, such as by capillary action. Analyte-magnetic component-reporter molecule complexes are concentrated in the portion of the capillary to which the magnetic field is applied 114, such as by a magnet or magnetic strip 116.

Further in FIG. 1, uncomplexed target analytes, magnetic components 104, reporter molecules 106, and control particles 110 flow laterally through the capillary 112. Control particles 110 can be bound in a control section 118 at or near the distal end of the capillary 112. An optional sample absorption section 120 is fluidly associated with the distal end of the capillary 112 and is configured to absorb the sample.

In another embodiment, as illustrated in FIG. 2 and described in more detail below, target analyte preparation can begin with a bacteriophage 200 that infects a bacteria cell 202. The phage undergoes phage amplification to produce high copy numbers of a target analyte 204. The analyte is loaded onto a detection unit 206 as described above for FIG. 1. The target analyte can be detected visually on the detection unit by color formation. Additionally or alternatively, the target analyte can be detected by a detection device 210. Additionally or alternatively, use of reporter molecules that are SERS reporter molecules surface-conjugated with anti-analyte antibodies allows for the elaboration of a quantifiable signal in the form of a predetermined Raman spectrum 212. The spectrum can be detected using a detection device 210 that is a handheld Raman spectrometer.

The systems, devices, and methods described herein can include any number of components described herein in any combination.

### Target Analytes

Target analytes can include any antigen known in the art, including proteins, prions, hormones, enzymes, cytokines, neurotransmitters, immunoregulatory molecules, cancer markers, toxins, chemicals, pharmaceuticals, viruses, bacteria, infectious agents, fungi, protozoa, algae, and cells.

In some embodiments, target analytes are a bacteriophage (phage) or viral agent that bind to and/or infect a bacterium or other microorganism. The phage or viral agent can be any molecule known in the art. The phage can undergo amplification. A phage can be a phage amplification product.

### Phage Amplification

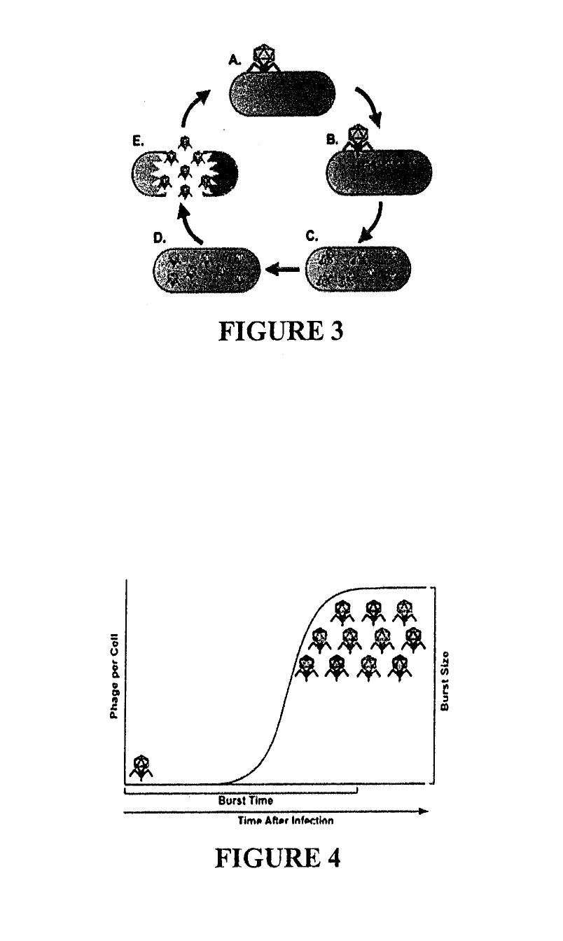

In certain embodiments, a target analyte that is a phage can be amplified prior to being combined with other sample components such as a magnetic component and/or a reporter molecule. FIG. 3 illustrates an exemplary infectious cycle. In step A), species-specific phage attachment to a bacterial cell is followed by insertion of phage genetic material into a host B). Transcription and translation of phage genes then occur using a combination of phage-encoded and host cellular machinery, which results in the production of numerous copies of progeny phage components C). Progeny phages are then assembled intracellularly D) followed by eventual lysis of the host bacterium E) releasing progeny phages for subsequent infection of remaining uninfected bacteria. Attachment of phage to a bacterial cell can be, for example, by markers known in the art such as Gram-positive peptidoglycan, Gram-negative outer membrane porins, transporter proteins, or by attachment to bacterial cell wall components such as lipopolysaccharide.

Depending on the phage-host pair, an infectious cycle can result in amplification rates ranging from a few hundred to several thousand new phages from each bacterial lysis event. FIG. 4 illustrates a classic growth plot demonstrating exponential amplification resulting from infection of a single bacterium with a single phage. Following the initial infection, the number of phages remains constant for a short time (the latent period). As infection progresses and progeny phages are released, additional infections occur resulting in a rapid rise in the number of free phages available for subsequent infection. This expansion levels off at many times the number of original infecting phages. The time from phage attachment to a cell until lysis of the cell and release of new phages is termed the burst time. The ratio of the number of phages present at the beginning of an infection to the number of phages present after an infection is termed the burst size.

In some embodiments, detection limits are lowered by several orders of magnitude by exploiting a large burst of progeny phage and focusing on species-specific phage detection rather than directly on the bacterial species of interest. In some embodiments, further sensitivity can be added by determining which phages have the best possible combination of large burst size and short burst time.

### Sample Loading Section

The systems, devices, and methods described herein can include a sample loading section configured to receive a sample. Receiving can be loading a sample onto the section. Receiving can be fluid movement that carries the sample to the section. Receiving can be any other method known in the art.

As described below, the sample loading section can be fluidly associated with one or more of the following: the proximal end of the capillary, a magnetic component loading section, a reporter molecule loading section, and a magnetic component-reporter molecule loading section.

### Magnetic Components

The systems, devices, and methods described herein can include at least one magnetic component. The magnetic component can be a molecule, a particle, a nanoparticle, or any other similarly small component. The magnetic component can be ferrimagnetic. The magnetic component can be configured to bind a target analyte. The surface of the magnetic component can be coated with anti-analyte antibodies. The magnetic component can be configured to bind a target analyte by any ligand-receptor interaction known in the art.

The magnetic component can be associated with a magnetic component loading section, which is fluidly associated with the proximal end of the capillary and fluidly associated with the sample loading section. In some embodiments, the magnetic component loading section is configured to receive at least one magnetic component. For example, a magnetic component can be loaded onto the magnetic component loading section. Alternatively, a fluid can carry the magnetic component to the magnetic component loading section. Any other method known in the art can be used to load a magnetic component onto a magnetic component loading section.

In some embodiments, the magnetic component loading section is configured to release at least one magnetic component. For example, a target analyte can bind the magnetic component and carry it in a fluid stream.

The magnetic component loading section may be a well, a pad, or any other medium. In some embodiments, the magnetic component loading section is prepared by soaking glass fiber media with magnetic component solutions. The media is then air dried in a sterile dessication chamber.

The magnetic component loading section can be of any size, shape, or density.

### Reporter Molecules

The systems, devices, and methods described herein can include at least one reporter molecule. Reporter molecules can bind a target analyte. Reporter molecules can bind a target analyte by any binding mechanism known in the art. In some embodiments the reporter molecule is conjugated to a receptor that interacts with a target analyte that is a ligand. In some embodiments, the reporter molecule is conjugated to an antibody that binds a target analyte that is an antigen.

In some embodiments, the reporter molecule is a SERS reporter molecule. SERS reporter molecules to which antibodies capable of binding target analytes are attached are described in U.S. patent application Ser. No. 12/351,522, filed Jan. 9, 2009, which is incorporated by reference herein in its entirety.

Raman spectroscopy provides a molecular level signature of a chemical species through coupling of incident photons with selected vibrational normal modes and subsequent collection of the scattered radiation. Attaching molecules of interest to metal surfaces provides an enhanced Raman signal due to coupling mechanisms that involve the polarizability of a molecule and the electric field that it experiences while in close proximity to a metal surface. This surface enhancement (i.e. surface-enhanced Raman spectroscopy (SERS)) can increase Raman signals by three to six orders of magnitude, making it a viable probe for target analytes present at low concentrations.

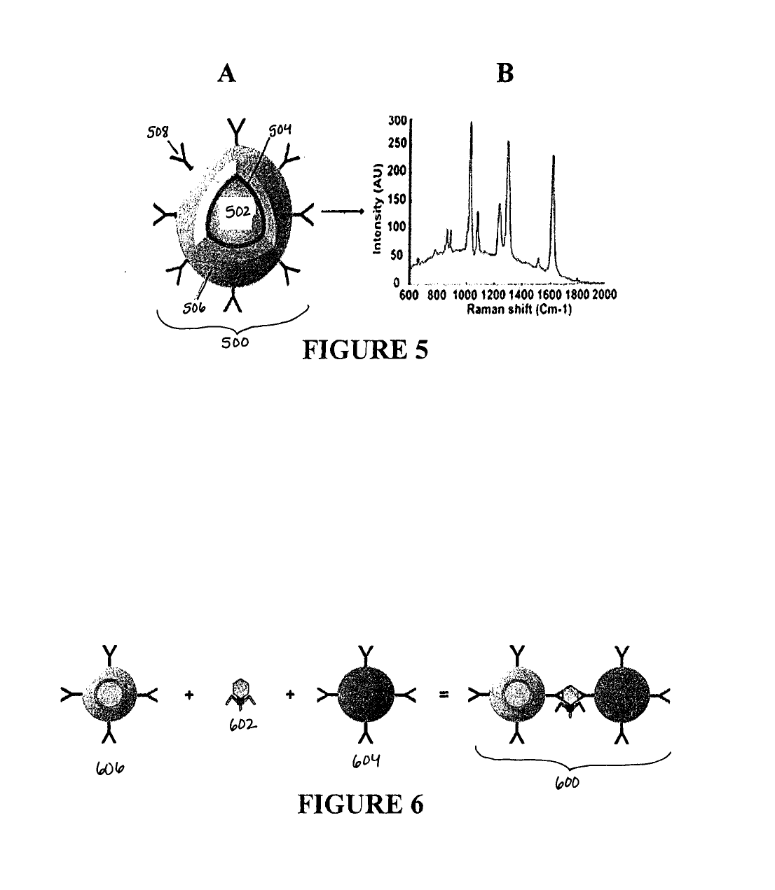

FIG. 5A depicts one embodiment of a reporter molecule that is a SERS reporter molecule 500 (Oxonica, Mountain View, Calif.). As shown in FIG. 5A, the SERS reporter molecule 500 includes a metal core 502, an organic reporter molecule coating 504, a glass encapsulation 506 and analyte-specific antibodies 508. In one embodiment, the molecule has a metal core 502, with an organic reporter molecule coating 504 attached to or in close proximity to the metal core 502 and a glass encapsulation 506 attached to the surface of the organic reporter molecule coating 504, and analyte-specific antibodies 508 attached to the surface of the glass encapsulation 506.

As illustrated in FIG. 5A, the depicted SERS reporter molecule 500 includes a metal core 502. The metal core 502 can be any metallic composition that is known in the art to have electromagnetic or chemical enhancement properties. In one embodiment, the metal core 502 is gold (Au). In another embodiment, the metal core 502 is colloidal gold. In an alternative embodiment, the metal core 502 is silver (Ag). The metal core 502 can also be copper (Cu), sodium (Na), potassium (K), chromium (Cr), aluminum (Al), lithium (Li), or a metal alloy. In other embodiments, the metal core 502 can be pure metal or a metal alloy and can be overlaid with at least one metal shell.

Referring again to FIG. 5A, the organic reporter molecule coating 504 is attached to the metal core 502. The organic reporter molecule coating 504 is a spectroscopy-active layer and exhibits a simple Raman spectrum (FIG. 5B). The Raman spectrum is enhanced when the organic reporter molecule coating 504 is in close proximity to a metal surface such as the metal core 502. A person skilled in the art will recognize that the organic reporter molecule coating 504 can be any type of molecule with a measurable SERS spectrum, and can be a single layer or multi-layered. A measurable spectrum is one in which the presence of the organic reporter molecule coating 504, and/or possibly the core, can be detected and recognized as a characteristic of the particular organic reporter molecule coating 504. Generally, suitable Raman-active organic reporter molecule coatings have (i) strong Raman activity thus minimizing the number of particles necessary to provide a detectable signal and (ii) a simple Raman spectrum which permits the use of multiple different particles which can be distinguished even if used simultaneously.

As shown in FIG. 5A, in one embodiment, analyte-specific antibodies 508 are located on an external surface of the SERS reporter molecule 500. The analyte-specific antibodies 508 can be grafted, bound or otherwise operably attached to an external surface of a SERS reporter molecule 500, i.e., onto the glass encapsulation 506. Use of non-specific analyte antibodies increases the likelihood of cross-reactivity to other analytes present in the sample. Thus, it is advantageous to use analyte-specific antibodies to reduce the likelihood of cross-reactivity, thereby increasing efficiency and reliability.

In some embodiments, the reporter molecule includes a visually detectable reporter molecule. A visually detectable reporter molecule can be conjugated to an optical dye. Optical dyes can include, but are not limited to fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Suitable optical dyes are described in the Sixth Edition of the Molecular Probes Handbook by Richard P. Haugland, hereby expressly incorporated by reference in its entirety; see chapters 1, 2 and 3 in particular.

In some embodiments, the reporter molecule includes a fluorescent reporter molecule. A fluorescent reporter molecule can be conjugated to a fluorescent label or fluorophore. Fluorescent labels include any molecule that can be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable fluorophores are described in Molecular Probes Handbook by Richard P. Haugland, hereby expressly incorporated by reference.

In some embodiments, a reporter molecule can include a proteinaceous fluorescent protein. Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Cum Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

In other embodiments, the reporter molecule includes a luminescent reporter molecule, a chemiluminescent, or an electrochemiluminescent reporter molecule. A luminescent reporter molecule, a chemiluminescent, or an electrochemiluminescent reporter molecule can be conjugated to or otherwise associated with a luminescent compound or label. An example of a luminescent compound or label includes, but is not limited to, luciferase, including a Renilla or Photinus species of luciferase.

In still other embodiments, the reporter molecule includes a phosphorescent reporter molecule. A phosphorescent reporter molecule can be conjugated to or otherwise associated with a phosphorescent label or compound. Examples of phosphorescent labels or compounds include, but are not limited to, eosin and eosin derivates such as eosin isothiocyantate.

The reporter molecule can be associated with a reporter molecule loading section, which is fluidly associated with the proximal end of the capillary and fluidly associated with the sample loading section. In some embodiments, the reporter molecule loading section is configured to receive at least one reporter molecule. For example, a reporter molecule can be loaded onto the reporter molecule loading section. Alternatively, a fluid can carry the reporter molecule to the reporter molecule loading section. Any other method known in the art can be used to load a reporter molecule onto a reporter molecule loading section.

In some embodiments, the reporter molecule loading section is configured to release at least one reporter molecule. For example, a target analyte can bind the reporter molecule and carry it in a fluid stream. The reporter molecule loading section can be a well, a pad, or any other medium. In some embodiments, the reporter molecule loading section is prepared by soaking glass fiber media with reporter molecule solutions. The media is then air dried in a sterile dessication chamber. The reporter molecule loading section can be of any size, shape, or density.

In some embodiments, the magnetic component loading section described above and the reporter molecule loading section are the same section (i.e. a magnetic component-reporter molecule loading section). In some embodiments, the magnetic component-reporter molecule loading section is fluidly associated with the proximal end of the capillary and fluidly associated with the sample loading section.

In some embodiments, the magnetic component-reporter molecule loading section is configured to receive and to release at least one magnetic component and at least one reporter molecule. Receipt and/or release can occur by any method described above for each of the magnetic component and reporter molecules alone.

### Analyte-Magnetic Component-Reporter Molecule Complexes

In some embodiments, a target analyte is mixed with at least one magnetic component and/or at least one reporter molecule. The mixing can occur actively, such as by combining a target analyte and a magnetic component and/or a reporter molecule in a tube, vial, or other vessel. The mixing can occur passively, such as by fluid movement that brings a target analyte and a magnetic component and/or a reporter molecule together. As shown in FIG. 6, when a target analyte 602, magnetic component configured to bind the target analyte 604, and reporter molecule configured to bind the target analyte 606 are mixed, they can form an analyte-magnetic component-reporter molecule complex 600 in a sample.

### Capillaries

The capillaries describe herein can substitute for any lateral flow device. The capillary can be constructed of any material known in the art that transmits light in the visible spectrum. In various embodiments, the capillary is constructed of a material that is not Raman-detectable. In other embodiments, the capillary is designed of a material that is clear such as glass, polymer, or plastic. In certain embodiments, the capillary is manufactured of borosilicate. In other embodiments, the capillary is manufactured of polycarbonate. In other embodiments, the capillary is manufactured of polycarbon glass. In still other embodiments, the capillary is manufactured of plastic polymer, polyethylene, polypropylene, polystyrene, polybutylene, or acrylic.

The capillary can be constructed in any shape. In some embodiments, the capillary is cylindrical with a curved (e.g. elliptical or circular) circumference. In other embodiments, the capillary has one or more flat, uncurved surfaces. In certain variations, the capillary has a rectangular circumference, or a square circumference.

The capillary can be constructed in various lengths and various diameters.

The capillary can be affixed to a solid support. The solid support can be plastic backing board.

In various embodiments, a sample is introduced to the capillary. The sample can be introduced actively, such as by loading the sample into the proximal end of the capillary. The loading can be done by a pipette. The loading can be done by injection with a microsyringe. The sample can be introduced passively, such as by fluid movement that delivers a sample into the capillary. The fluid movement can be capillary action. The proximal end of the capillary can be fluidly associated with the sample loading section.

### Magnetic Fields

The systems, devices, and methods described herein can include a magnetic field. The magnetic field can be applied to at least a portion of the capillary. The magnetic field can be applied anywhere along the length of the capillary. The magnetic field can be applied midway along the length of the capillary.

The magnetic field can be created by a magnet or magnetic strip. The magnetic strip can be neodymium. The magnet or magnetic strip can be positioned outside of the capillary. The magnet or magnetic strip can be positioned above or below the capillary. The magnet or magnetic strip can be positioned anywhere along the length of the capillary, including midway along the length of the capillary. The magnet or magnetic strip can be affixed to or embedded in a solid support or it can be free-standing.

If an analyte-magnetic component-reporter molecule complex is present in a sample, it can be directed to the portion of the capillary to which the magnetic field is applied. If more than one analyte-magnetic component-reporter molecule complex is present, the complexes can be concentrated at the portion of the capillary to which the magnetic field is applied.

### Sample Absorption Sections

In some embodiments, a sample absorption section is fluidly associated with the distal end of the capillary. The sample absorption section can be configured to absorb a sample. The sample absorption section can be a well, a pad, a paper, or any other media. The section can be of any size, shape, or density.

In some embodiments, the sample absorption section can also be configured to wick a sample through the capillary. In some embodiments, the section can also be configured to indicate the presence of a control particle, as described below.

### Detection Units

In some embodiments, as illustrated in FIG. 1, some or all of the sample loading section, magnetic component-reporter molecule loading section, capillary, magnet or magnetic strip, control section, and sample absorption section make up a detection unit. These components can be associated to create an interlocking platform. In other embodiments, the components can be affixed to or embedded in one or more solid supports.

### Detectors and Detection

Analyte-magnetic component-reporter molecule complexes concentrated in the portion of the capillary to which a magnetic field is applied result in the formation of detectable complexes. In some embodiments, the concentrated complexes can form a detectable line.

In some embodiments, the detection can be of an optical dye conjugated to a reporter molecule. In some embodiments, the detection can be visual. A detector can be at least one human eye. A detector can be a densitometer.

In some embodiments, the detection can be of a fluorescent label or a fluorophore conjugated to a reporter molecule. A detector can be a fluorometer.

In some embodiments, the detection can be of a luminescent label conjugated to a reporter molecule. In other embodiments, the detection can be of a chemiluminescent or electrochemiluminescent label conjugated to a reporter molecule. A detector can be a luminometer.

In some embodiments, the detection can be of a phosphorescent label conjugated to a reporter molecule. A detector can be a phosphorimeter.

In some embodiments, detection can be by spectrometry, and a detector can be a spectrometer. As previously described for FIG. 5, when the reporter molecule is a SERS reporter molecule 500, the organic reporter molecule coating 504 of a SERS reporter molecule 500 is a spectroscopy-active layer that exhibits a simple Raman spectrum (FIG. 5B). An analyte-magnetic component-reporter molecule complex can thus also be detected by Raman spectrometry, or a detector can be a Raman spectrometer. In some embodiments, the Raman spectrometer is a benchtop model. In other embodiments, the Raman spectrometer is handheld.

The Raman spectrometer can be a commercially available Raman spectrometer. Examples of commercially available Raman spectrometers include, but are not limited to, Raman spectrometers from the following companies: DeltaNu (Laramie, Wyo.), Thermo, Rigaku, Perkin Elmer, Ocean Optics, Bruker, Enwave, and Lambda Solutions.

While Raman spectrometers are used in various embodiments, any form of monochromator or spectrometer that can temporally or spatially resolve photons and any type of photon detector known in the art can be used.

Detection of a complex can indicate the presence of a target analyte. Detection of a complex can also serve as a positive test for the presence of a target analyte. Detection of an analyte-magnetic component-reporter molecule complex by any method described above can indicate the presence of a bacteria that has been subject to phage infection and amplification.

A microorganism can be detected by detecting a target analyte, wherein the presence of the target analyte corresponds to the presence of a microorganism.

In some embodiments, less than 1,000,000 target analytes can be detected. In other embodiments, less than 500,000 target analytes can be detected. In other embodiments, less than 100,000 target analytes can be detected. In other embodiments, less than 10,000 target analytes can be detected. In other embodiments, less than 1,000 target analytes can be detected. In other embodiments, less than 500 target analytes can be detected. In other embodiments, less than 100 target analytes can be detected. In other embodiments, less than 50 target analytes can be detected. In other embodiments, less than 10 target analytes can be detected. In other embodiments, 1 target analyte can be detected. In other embodiments, the lower detection limit is between 100 and 1,000 target analytes. In other embodiments, the lower detection limit is between 1 and 100 target analytes.

In some embodiments, less than 1,000,000 colony forming units (cfu)/mL can be detected. In other embodiments, less than 500,000 cfu/mL can be detected. In other embodiments, less than 100,000 cfu/mL can be detected. In other embodiments, less than 10,000 cfu/mL can be detected. In other embodiments, less than 1,000 cfu/mL can be detected. In other embodiments, less than 500 cfu/mL can be detected. In other embodiments, less than 100 cfu/mL can be detected. In other embodiments, less than 50 cfu/mL can be detected. In other embodiments, less than 10 cfu/mL can be detected. In other embodiments, 1 cfu/mL can be detected. In other embodiments, the lower detection limit is between 100 and 1,000 cfu/mL. In other embodiments, the lower detection limit is between 1 and 100 cfu/mL.

In some embodiments, the target analyte is a phage and the concentration of detected target analytes is measured as plaque forming units (pfu)/mL. This value can then be converted to colony forming units cfu/mL by dividing the pfu/mL value by the known phage burst size. The resulting cfu/mL value expresses bacterial concentration indirectly detected by phage amplification.

### Control Particles

In certain embodiments, a control particle can be used to indicate whether a sample successfully traverses all or a portion of the capillary. The control particle can be used alone as its own sample or in conjunction with a target analyte. If used in conjunction with a target analyte, it can be added before or during the mixing of the target analyte, magnetic component, and/or reporter molecule.

The control particle can be or can be conjugated to a nanoparticle, a molecule, a native molecule, a recombinant molecule, a synthetic molecule, a small molecule, an enzyme, a peptide, a peptide subunit, an aptamer, a lectin, a complex, a conjugate, a whole organism, or any other similarly small particle known in the art.

The control particle can enter the capillary and travel to or through the distal end of the capillary. The control particle can be arrested at or near the distal end of the capillary. In some embodiments, the control particle is arrested at a control section, which is fluidly associated with the distal end of the capillary. In some embodiments, the control section is configured to bind a control particle.

Any receptor-ligand interaction known in the art can be used to arrest control particles in, for example, a control section or a sample absorption section. In some embodiments, a control particle is surface-coated with biotin. The distal internal portion of the capillary can be coated with avidin. Alternatively, a sample absorption section fluidly associated with the distal end of the capillary can be coated with avidin. The particle is bound when the biotin comes in contact with the avidin. The avidin can be streptavidin. The control particle can be a red polystyrene nanoparticle surface-conjugated with biotin.

In other embodiments, the control particle is surface-coated with an antigen. The distal internal portion of the capillary can be coated with an antibody that binds the antigen. Alternatively, a sample absorption section fluidly associated with the distal end of the capillary can be coated with an antibody that binds the antigen. The particle is bound when the antigen comes in contact with the antibody.

In other embodiments, the control particle is a whole organism that is arrested by anti-organism antibodies that are immobilized at a control section. For example, the control particle can be Escherichia coli bound to a colored particle, and anti-E. coli antibodies can be the receptors that bind to the E. coli and arrest their movement.

In still other embodiments, the control particle is or is conjugated to a monosaccharide or oligosaccharide that is arrested by a lectin molecule at a control section. In still other embodiments, a control particle is conjugated to the enzyme horseradish peroxidase and the particle is arrested by interacting with its substrate which is immobilized at a control section.

Arrested control particles can become concentrated at or near the point of arrest, producing detectable particles. The detection may be visual. The particles can produce a detectable line or region. The detectable line or region can be a colored line or region. The colored line or region can be red.

Detection of a control particle can be by any means known in the art. Detection of a control particle can indicate that a sample successfully traverses most or all of the length of the capillary. Detection of a control particle can indicate that a sample is successfully transported to the distal end of or through the capillary.

### Multiplexed Systems and Devices

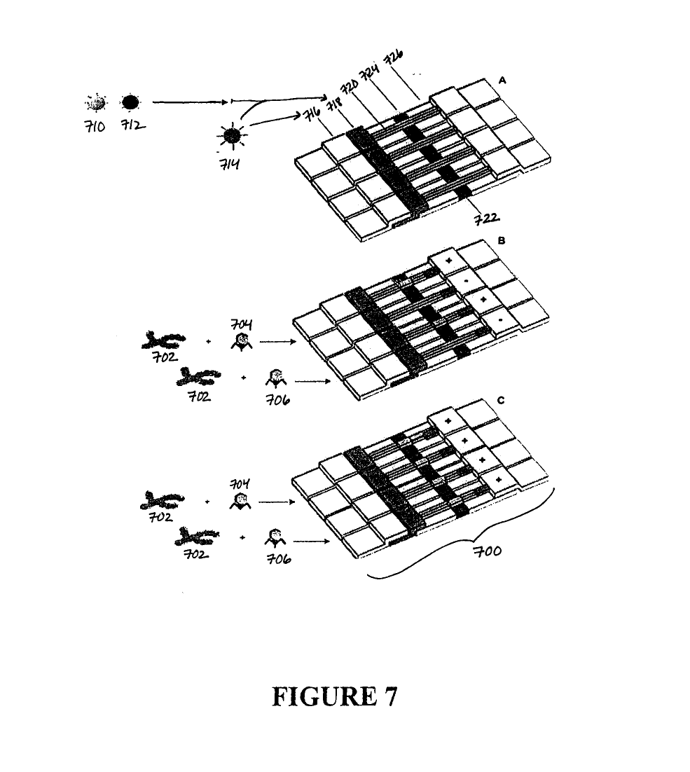

In some embodiments, as depicted in FIG. 7 and as described in detail below, the system or device can be multiplexed. Each individual detection unit operates as described above for FIG. 1. Each detection unit can serve as a test for the presence of a different analyte.

In some embodiments, a reporter molecule is a SERS reporter molecule. In those embodiments, a SERS reporter molecule with a unique organic reporter molecule coating, which produces a distinct Raman spectrum, is designed and used for each type of analyte.

### Kits

In some embodiments, any two or more of the components described above can comprise a kit. A kit can include any number of components described above in any combination. The components can be assembled or unassembled. In some embodiments, the kit includes instructions for assembling or using the components.

### EXAMPLES

The following examples illustrate various aspects of the disclosure, and should not be considered limiting.

### Example 1

### SERS-Mediated Bacterial Detection

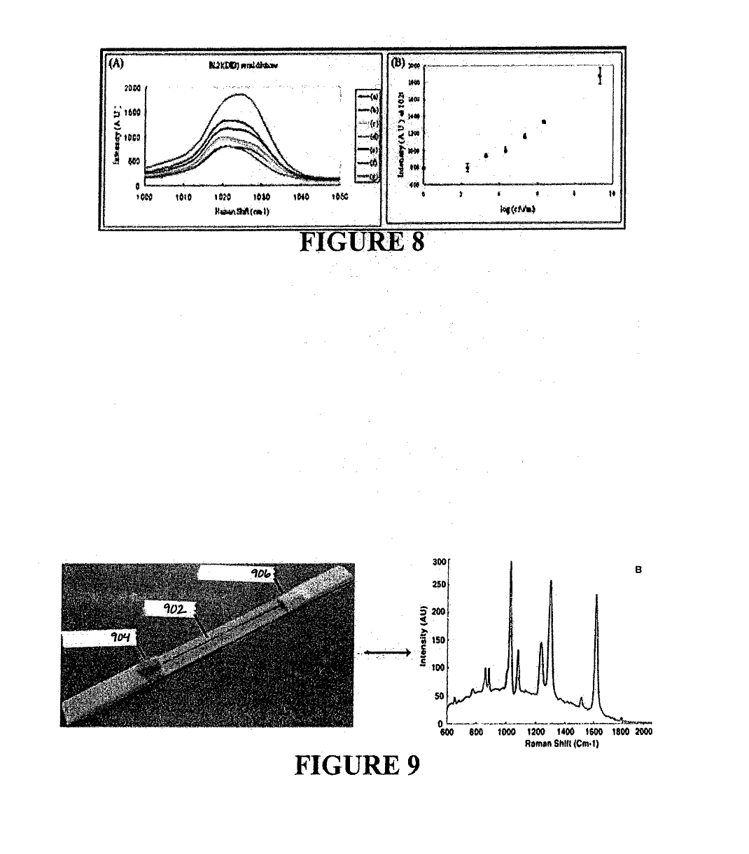

Studies using conventional lateral flow immunochromatography resulted in a visual limit of detection (LOD) of 10⁶ cfu/mL. Studies using E. coli and MS2, an E. coli-specific phage, were designed to assess the feasibility of improving this LOD with SERS. These studies suggest the LOD can be reduced to a range between 10² to 10³ cfu/mL (FIG. 8). FIG. 8A depicts the spectra of a, negative control, 0 cfu/mL; b, 2'10² cfu/mL; c, 2×10³ cfu/mL; d, 2×10⁴ cfu/mL; e, 2×10⁵ cfu/mL; f, 2×10⁶ cfu/mL; and g, 2×10⁹ cfu/mL. FIG. 8B depicts a titration curve, which demonstrates a high correlation between Raman signal sensitivity and E. coli concentration.

The greatest reduction in LOD is obtained with optimization of study conditions and components including the magnetic components, the SERS reporter molecules, and the antibody concentration on the SERS reporter molecules.

### Example 2

### SERS Interrogation of a Borosilicate Capillary

A detection unit was fabricated using a 2 μl-capacity glass capillary 902 and conventional sample loading 904 and absorbing 906 media (FIG. 9A). A colloidal solution of SERS reporter molecules was applied to the device and analyzed by Raman spectroscopy. A strong Raman signal was observed (FIG. 9B). The relative signal strength derived from using capillary-based devices was significantly enhanced as compared to conventional membrane-based devices.

### Example 3

### Bacterial Identification and Antimicrobial Resistance Determination

Enterococci are Gram-positive cocci; two species, Enterococcus faecalis and E. faecium are leading causes of nosocomial infections. Some enterococci are susceptible to antibiotics such as vancomycin (VSE) and others are resistant (VRE).

VSE and VRE E. faecalis and E. faecium are detected and identified using multiplexed detection as depicted in FIG. 7. A sample from a patient (e.g. feces, urine, serum, skin swab, etc.) 702, or an environmentally-derived material (e.g. hospital surface swab) 702, or other wet or dry material suspected of containing Enterococcus 702 is mixed with two sets of previously prepared phage infection vials. One set is comprised of a vial containing E. faecalis-specific phage(s) 704 and a nutritive broth optimized to facilitate phage infection and propagation. The other vial contains phage and nutritive broth containing vancomycin. The second set is comprised of a vial containing E. faecium-specific phage(s) 706 and nutritive broth and a second vial containing phage and nutritive broth containing vancomycin. Two separate tests are required for each phylotype (one with vancomycin and one without) because VSE is killed by vancomycin, which precludes phage amplification (PA). This would result in a false negative test when a sample is actually positive for VSE.

Samples are incubated for one to two hours to allow for phage amplification. Following incubation, small aliquots of the individual PA reactions are applied to multiplexed Enterococcus detection units 700 where the liquid samples wick from the sample loading sections 716 into a magnetic component-reporter molecule loading section 718 containing magnetic components 710 and SERS reporter molecules 712 (Oxonica, Mountain View, Calif.), both surface-conjugated with anti-analyte antibodies, and a control particle 714 consisting of red polystyrene nanoparticles surface-conjugated with biotin (FIG. 7A). Analyte-magnetic component-reporter molecule complexes, as well as control particles 714, enter the capillary 720. The sample then travels toward a magnet 722 outside of the capillary 720.

Analyte-magnetic component-reporter molecule complexes are arrested and concentrated in the capillary 720 at the location of the magnetic field 724 produced by the magnet 722. Concentrated complexes result in the formation of a pink line due to the pink color of the SERS reporter molecules (FIGS. 7B and 7C). The presence of E. faecalis or E. faecium can be visually detected by this line.

At the same time as the complexes, the control particle 714 is carried to an immobilized stripe of avidin coating the distal inside wall of the capillary. The biotin-conjugated control particle 714 is concentrated at the distal end of the capillary, called a control section 726, resulting in the formation of a visible red line, indicating that the sample was successfully transported through the length of the capillary 720 (FIGS. 7B and 7C).

A positive result is indicated by the formation of pink and red lines at the magnetic field 724 and control section 726, respectively. A negative result is observed as the formation of a red line at the control section 726 only. If an Enterococcus-positive sample is vancomycin sensitive only the Enterococcus detection test will be positive (FIG. 7B). If a positive sample is vancomycin resistant both capillaries will be positive (FIG. 7C).

In addition to the unaided visual detection of a color formation on the detection unit, the use of SERS reporter molecules surface-conjugated with anti-analyte antibodies allows for the elaboration of a quantifiable signal in the form of a predetermined Raman spectrum. The spectrum can be detected using a handheld Raman spectrometer.

### Example 4

### Bacterial Identification

In another example, detection tests for Yersinia pestis, Bacillus anthracis, Burkholderia mallei and Burkholderia pseudomallei are multiplexed, and include a phage-only positive control. All steps are performed as described in Example 3. A positive test is indicated by the formation of pink and red complexes at the magnetic field and control section, respectively. A negative result is observed as the formation of a red complex at the control section only.

The above specification and examples provide a complete description of the structure and use of exemplary embodiments of the invention. Although various embodiments of the invention have been described above with a certain degree of particularity, or with reference to one or more individual embodiments, those skilled in the art could make numerous alterations to the disclosed embodiments without departing from the spirit or scope of this invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A system for detecting a target analyte comprising:
a sample loading section configured to receive a sample;
a capillary in which the proximal end of the capillary is fluidly associated with the sample loading section;
a magnet configured to apply a magnetic field to at least a portion of the capillary; and a detector configured to detect an analyte-magnetic component-reporter molecule complex in the capillary.

2. The system of claim 1, further comprising a solid support to which the capillary is affixed.

3. The system of claim 1, further comprising a magnetic component-reporter molecule loading section fluidly associated with the proximal end of the capillary and fluidly associated with the sample loading section, and configured to receive and to release at least one magnetic component and at least one reporter molecule to said target analyte.

4. The system of claim 1, further comprising a magnetic component loading section fluidly associated with the proximal end of the capillary and fluidly associated with the sample loading section and configured to receive and to release at least one magnetic component, and a reporter molecule loading section fluidly associated with the proximal end of the capillary and fluidly associated with the sample loading section and configured to receive and to release at least one reporter molecule.

5. The system of claim 3, wherein the at least one magnetic component is surface-coated with anti-analyte antibodies.

6. The system of claim 3, wherein the at least one reporter molecule is a SERS reporter molecule.

7. The system of claim 6, wherein the SERS reporter molecule comprises a colloidal gold core coated with an organic reporter molecule encapsulated in glass to which anti-analyte antibodies are bound.

8-9. (canceled)

10. The system of claim 1, further comprising a control section fluidly associated with the distal end of the capillary and configured to bind a control particle.

11. The system of claim 10, wherein the control particle is a red polystyrene nanoparticle surface-conjugated with biotin.

12. (canceled)

13. The system of claim 1, wherein the target analyte is a bacteriophage.

14-19. (canceled)

20. The system of claim 1, wherein the detector is at least one human eye, a Raman spectrometer, a densitometer, a fluorometer, a luminometer, or a phosphorimeter.

21-23. (canceled)

24. A method for detecting a target analyte comprising:
mixing a target analyte, a magnetic component configured to bind the target analyte, and a reporter molecule configured to bind the target analyte to form analyte-magnetic component-reporter molecule complex in a sample;
introducing the sample to a capillary;
applying a magnetic field to at least a portion of the capillary to concentrate the complex; and
detecting the complex to determine the presence of the target analyte.

25. The method of claim 24, wherein the target analyte is a bacteriophage.

26. (canceled)

27. The method of claim 24, further comprising adding a control particle before or during said mixing step.

28. (canceled)

29. The method of claim 24, wherein the magnetic component is surface-coated with anti-analyte antibodies.

30. The method of claim 24, wherein the reporter molecule is a SERS reporter molecule.

31. The method of claim 30, wherein the SERS reporter molecule comprises a colloidal gold core coated with an organic reporter molecule encapsulated in glass to which anti-analyte antibodies are bound.

32-36. (canceled)

37. The method of claim 24, wherein the magnetic field is applied to a portion of the capillary midway along the length of said capillary.

38. (canceled)

39. The method of claim 24, wherein the detecting is by a Raman spectrometer, a densitometer, a fluorometer, a luminometer, or a phosphorimeter.

40-44. (canceled)

45. The method of claim 24, wherein the detecting has a lower detection limit of between 1 and 100 cfu/mL.

46. (canceled)
